(19) United States
(12) Patent Application Publication
(10) Pub. No.: US 2015/0036103 A1
(43) Pub. Date: Feb. 5, 2015

(54) TRANSMISSION DIFFRACTIVE OPTICAL ELEMENT AND MEASURING DEVICE

(71) Applicant: CANON KABUSHIKI KAISHA, Tokyo (JP)

(72) Inventor: Tsuyoshi Kitamura, Utsunomiya-shi (JP)

(21) Appl. No.: 14/444,772

(22) Filed: Jul. 28, 2014

(30) Foreign Application Priority Data
Jul. 30, 2013 (JP) 2013-157609

(51) Int. Cl. G02B 5/18; A61B 3/12; A61B 3/10

(52) U.S. Cl. CPC G02B 5/1866; A61B 3/102; A61B 3/12; G02B 2005/1804
USPC 351/206; 359/569

(57) ABSTRACT

A transmission diffractive optical element formed of quartz for a wavelength of 0.8 μm band, wherein a refractive index of the quartz is n, a wavelength of light entering a diffraction grating is λ (nm), a pitch of the diffraction grating is p (nm), a depth of the diffraction grating is D (μm), and a duty ratio in which a width of the diffraction grating is divided by the pitch is α, wherein the pitch and the depth and the duty ratio of the diffraction grating satisfy a predetermined condition for acquiring high diffraction efficiency.

### BACKGROUND OF THE INVENTION

1. Field of the Invention

The present invention relates to a transmission diffractive optical element and a measuring device.

2. Description of the Related Art

An optical coherence tomography (OCT) has been used for examining a fundus. A spectral-domain OCT (SD-OCT) is known as one of such systems in the OCT. The SD-OCT detects the frequency distribution of light from a subject using a light source emitting a wide band wavelength light and a spectroscope and performs Fourier transform of the detection signal to acquire a tomographic image in a depth direction. In the spectroscope a diffractive optical element separates interfering light between reference light and subject light from the fundus for each wavelength and a sensor detects light intensity (light quantity) for each wavelength. The OCT for examining the fundus uses wavelengths of 0.8 μm band as the wavelength of the light source, for example, a wide band of the center wavelength of 840 nm to 880 nm.

The diffraction efficiency of the diffractive optical element in the OCT affects the brightness of a sectional image to be acquired. A decrease in the diffraction efficiency at the edge of a wavelength band to be used leads to a deterioration in resolution in the sectional image. This is because the resolution is inversely proportional to the half width of a spectrum, so that a difference in the diffraction efficiency between a center wavelength and a wavelength at the edge of the wavelength band decreases the half width of the spectrum and increases (or deteriorates) the resolution. Therefore, it is important that the diffraction efficiency is higher not only in the center wavelength but also in the total area of the wavelength band in which the diffractive optical element performs spectroscopy. Since random polarized light enters the diffractive optical element, a high diffraction efficiency to both of transverse electric (TE) and transverse magnetic (TM) polarization is required of the diffractive optical element.

A transmission diffractive optical element emitting mainly low-order diffracted light is generally higher in the diffraction efficiency among various types of diffractive optical elements. U.S. Pat. No. 6,747,799 discusses conditions for a pitch, width, and height of a diffraction grating for realizing a diffraction efficiency of 90% or higher for both of the TE and the TM polarization of a conventional (C) band (1530 nm to 1565 nm) for optical communication with respect to high diffraction efficiency of a transmission diffractive optical element. Japanese Patent No. 4749789 discusses a technique in which the diffraction grating a thin film layer is provided immediately under the diffraction grating and a refractive index distribution of the thin film layer is brought into a distribution suited for the diffraction grating realizing a high diffraction efficiency. The wavelength of light entering the diffractive optical element according to Japanese Patent No. 4749789 is 1350 nm to 1750 nm.

U.S. Pat. No. 6,747,799 discusses a condition for a grating for realizing a high diffraction efficiency for both of the TE and the TM polarization in the C-band for optical communication. However, the condition does not realize a high diffraction efficiency for both of the TE and the TM polarization at a wavelength of 0.8 μm band.

The wavelength of light entering the diffractive optical element according to Japanese Patent No. 4749789 is 1350 nm to 1750 nm. The diffractive optical element according to Japanese Patent No. 4749789 does not realize a high diffraction efficiency for both of the TE and the TM polarization at a wavelength of 0.8 μm band either. Furthermore, a manufacturing process for forming the thin film layer according to Japanese Patent No. 4749789 is complicated and the production cost therefore increases. It is simpler to form the diffractive optical element by a single material without forming a thin film layer.

As stated above, a condition for the diffraction grating of the transmission diffractive optical element realizing a high diffraction efficiency for both of the TE and the TM polarization at a wavelength of 0.8 μm band has not been known.

### SUMMARY OF THE INVENTION

According to an aspect of the present invention, there is provided a transmission diffractive optical element formed of quartz for a wavelength of 0.8 μm band, wherein a refractive index of the quartz is n, a wavelength of light entering a diffraction grating is λ (nm), a pitch of the diffraction grating is p (nm), a depth of the diffraction grating is D (μm), and a duty ratio in which a width of the diffraction grating is divided by the pitch is α, wherein the pitch and the depth and the duty ratio of the diffraction grating satisfy the following equations:

2n×p<3λ,

D>7.8638×10^{-6×}p²−1.4279×10^{-2×}p+7.9734α>−8.5747×10⁻⁴×p+1.2328,

D>13.19×α²−14.16×α+5.360, and

D<15.44×α²−15.73×α+5.870.

Further features of the present invention will become apparent from the following description of exemplary embodiments with reference to the attached drawings.

### BRIEF DESCRIPTION OF THE DRAWINGS

FIG. 1 illustrates parameters of a diffractive optical element.

FIG. 2 is a schematic diagram illustrating the diffraction of a diffractive optical element.

FIGS. 3A, 3B, and 3C illustrate calculation results of diffraction efficiency at the time when the number of grooves of a diffraction grating is 1200.

FIGS. 4A, 4B, and 4C illustrate calculation results of diffraction efficiency at the time when the number of grooves of a diffraction grating is 1400.

FIGS. 5A, 5B, 5C, and 5D illustrate calculation results of diffraction efficiency at each number of grooves.

FIG. 6 is a chart illustrating a grating depth and a duty ratio which define straight lines A and B.

FIG. 7 is a chart illustrating curves C and D.

FIG. 8 illustrates results of calculation of diffraction efficiency at a predetermined wavelength band of a diffractive optical element according to a first exemplary embodiment.

FIG. 9 is a diagram illustrating the slanted side faces of the convex portions of a diffraction grating according to a second exemplary embodiment.

FIG. 10 illustrates results of calculation of diffraction efficiency at a predetermined wavelength band according to the second exemplary embodiment.

FIG. 11 is a schematic diagram illustrating an optical coherence tomography measuring device according to a third exemplary embodiment.

### DESCRIPTION OF THE EMBODIMENTS

Various exemplary embodiments, features, and aspects of the invention will be described in detail below with reference to the drawings.

A first exemplary embodiment describes a diffractive optical element used for an optical coherence tomography (OCT) for examining a fundus.

The OCT used for examining a fundus can acquire a sectional image and a stereoscopic image in various places on an eye ball such as a retina, anterior eye cornea, angle, and iris and has an excellent effect particularly in diagnosis of retinal disease such as macular degeneration and glaucoma. The OCT widely used in recent years is referred to as a spectral domain (SD) OCT. The SD-OCT detects the frequency distribution of light from a subject using a light source emitting a wide band wavelength light and a spectroscope and performs Fourier transform of the detection signal to acquire a tomographic image in a depth direction. In the spectroscope a diffractive optical element splits (disperses) interfering light between reference light and subject light from the fundus for each wavelength and a charge-coupled device (CCD) sensor detects light intensity (light quantity) for each wavelength. The diffractive optical element realizes spectroscopy such that light with a specific wavelength among light with a certain wavelength bandwidth that has entered a diffraction grating is transmitted or reflected in a specific direction using a diffraction phenomenon of light. The OCT used for examining a fundus uses wavelengths of 0.8 μm band as the wavelength of a light source, for example, a wide band of the center wavelength of 840 nm to 880 nm. For this reason, a diffractive optical element adapted to wavelengths of 0.8 μm band is used in the spectroscope. The system gains an advantage in speed of measurement and image processing, a spatial resolution, and a detection sensitivity in comparison with a time domain (TD) OCT for acquiring a sectional image in the depth direction of a detection object while mechanically moving a reference mirror of an interferometer.

One of performances required for the diffractive optical element in considering the performance of an image acquired by an OCT is a diffraction efficiency. The diffraction efficiency is a value indicating the ratio of the quantity of light diffracted to a desired order as to the quantity of incident light. Because the diffraction efficiency contributes to the brightness of an image, it is desirable that the diffraction efficiency is high.

The diffraction efficiency includes a polarization dependability of incident light. Light from a light source emitting light with a wide-band wavelength for the SD-OCT has generally no specific vibration direction with respect to a polarization. Therefore, it is desirable that the diffractive optical element has a high diffraction efficiency for both of the TE and the TM polarization (s polarization and p polarization).

The diffraction efficiency differs according to the wavelength of incident light. For example, even if the diffraction efficiency is high in a specific center wavelength and if the diffraction efficiency is low in a wavelength at the end of a wide wavelength band, the resolution of an image acquired after performing Fourier transform on a detection signal detected by the CCD sensor is deteriorated to lower measurement accuracy. For this reason, the diffraction efficiency needs to be high not only in a specific wavelength but also in at least all the wavelength band to be used.

In general, a transmission diffractive optical element is used as the diffractive optical element for the SD-OCT. This is because the transmission diffractive optical element is generally higher in diffraction efficiency than a reflection diffractive optical element. A diffractive optical element formed by performing directly etching onto a silica glass substrate with high purity is known as one configuration of the transmission diffractive optical element. Little light is lost on a surface opposite to the surface where a diffraction grating is formed because a reflection prevention film is coated on the surface opposite to the surface where the diffraction grating is formed. For this reason, almost all of losses in light in the transmission diffractive optical element are equal to light propagated by the diffraction grating at other orders excluding a desired order. On the other hand, a metal film coating with a reflection rate of approximately 90% is a reflection layer in the reflection diffractive optical element and losses in light in the reflection layer are great, so that the reflection diffractive optical element is lower in diffraction efficiency than the transmission diffractive optical element.

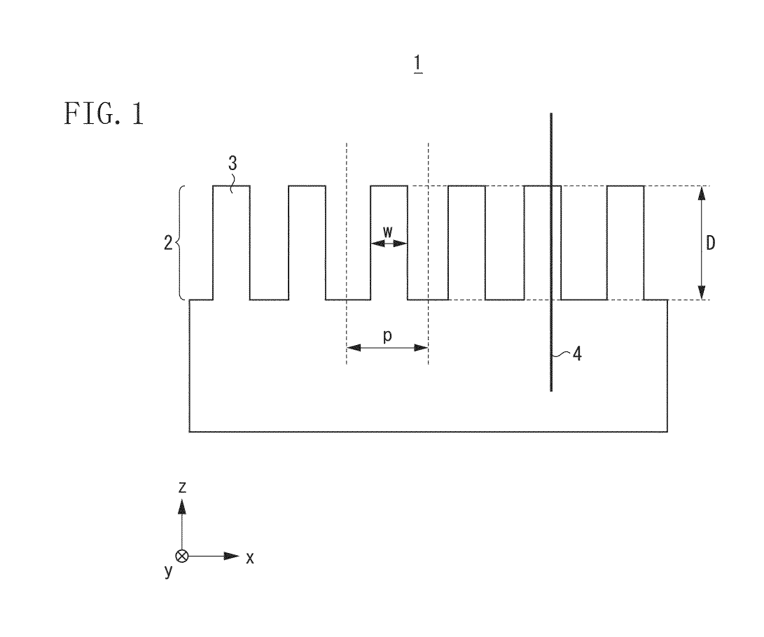

FIG. 1 is a cross section of a transmission diffractive optical element 1 according to the present exemplary embodiment. The transmission diffractive optical element 1 is formed such that a resist coated on a silica glass substrate is exposed by an exposure device and the exposed substrate is developed and etched. This method can form the transmission diffractive optical element by a simpler process than forming a thin film layer according to Japanese Patent No. 4749789. A diffraction grating 2 whose cross section is uneven is formed in the transmission diffractive optical element 1. The diffraction grating 2 is formed of a plurality of convex portions (convex portion 3) and ditches between the convex portions. In the present exemplary embodiment, the convex portion 3 of the diffraction grating 2 is rectangular. The convex portion 3 in the same sectional shape extends in the y direction, and a plurality of the convex portions 3 is repetitively arranged in the x direction to form a line and space diffraction grating 2.

Parameters defining the shape of the diffractive optical element 1 illustrated in FIG. 1 will be described below. The convex portions 3 of the diffraction grating 2 and the ditches between the convexes 3 are arranged in one direction at an equally spaced pitch p to diffract incident light. A reciprocal of the pitch p of the diffraction grating is referred to as the number of the ditches and usually indicated by the number of the ditches per millimeter. The length of the diffraction grating 2 (the convex portion 3) in the direction of a grating normal 4 (z direction) perpendicular to a diffraction grating surface on which the diffraction grating 2 is formed is defined as a grating depth D. The length of the convex portion 3 in the direction in which the convex portions 3 of the diffraction grating 2 are repetitively arranged (x direction) is defined as a grating width w. A value (w/p) in which the grating width w is divided by the pitch p is referred to as a duty ratio α. The shape of the diffraction grating 2 is uniquely determined by defining the pitch p or the number of the ditches, the grating depth D, and the duty ratio α.

The pitch p or the number of the ditches, the grating depth D, and the duty ratio a decide the order to which diffraction light is allocated, that is, significantly affect the diffraction efficiency. Therefore, by appropriately setting the pitch p or the number of the ditches, the grating depth D, and the duty ratio a, a high diffraction efficiency can be realized. In the present exemplary embodiment, the three parameters of the pitch p or the number of the ditches, the grating depth D, and the duty ratio α were changed by simulation using a computer to calculate the diffraction efficiency. The calculation is performed using a rigorous coupled-wave analysis (RCWA) method which is a type of an electromagnetic field analysis method.

The wavelength of incident light needs to be defined for the calculation. Because the light source of the SD-OCT practically applied at present has a wavelength band of a so-called 0.8 μm band whose center wavelength is 840 nm to 880 nm, the diffraction efficiency was calculated for the light of a specific wavelength 855 nm in this wavelength band in the present exemplary embodiment. The material for forming the diffractive optical element was a single material of silica glass. The silica glass is very high in transmissivity in a wavelength of 0.8 μm band, excellent in chemical stability, and inexpensive. It has been well known that silica glass is used as a transmission diffractive optical element such that a diffraction grating structure is provided by performing etching on a surface of the silica glass.

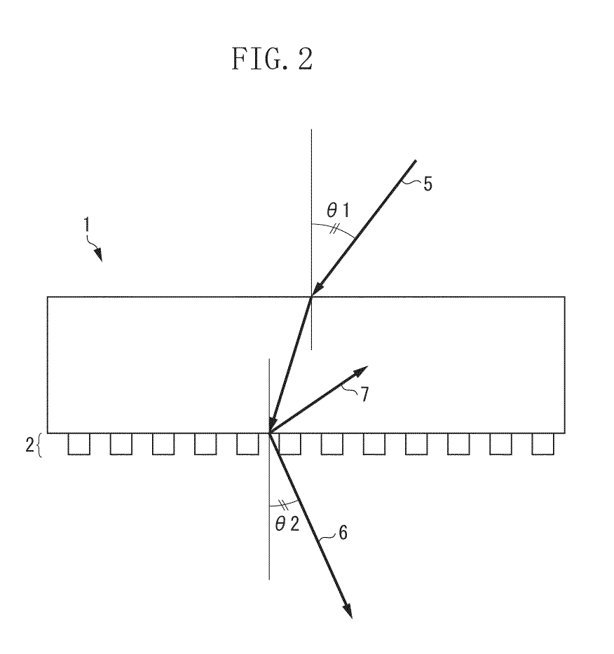

Incident and emitting angles of light were calculated in Littrow arrangement (condition). As illustrated in FIG. 2, incident light 5 enters the surface opposite to the surface on which the diffraction grating 2 is formed, transmitted by the diffractive optical element 1, diffracted by the diffraction grating 2, and outgoes. The Littrow arrangement refers to an arrangement in which an incident angle θ1 of the incident light 5 is equal to an emitting angle θ2 of a diffracted light 6 of a desired order. Since the emitting angle of the diffracted light differs according to the wavelength thereof, a device is designed such that the Littrow arrangement holds true for a certain wavelength, for example, the center wavelength in a wavelength band to be used.

The relationship between the incident angle θ1 of the incident light 5 and the emitting angle θ2 of the diffracted light 6 can be expressed by an equation of m×λ=p×(sin θ1+sin θ2), where a wavelength is λ(nm), a diffraction order is m, and a pitch of the diffraction grating 2 is p(nm). In the Littrow arrangement, an equation of m×λ=2p×sin θ is established when θ1 is equal to θ2 and a Littrow angle θis defined as θ=θ1=θ2.

In the present exemplary embodiment, a diffraction efficiency related to a primary diffracted light was calculated with the diffracted light 6 of the desired order as the primary diffracted light. This is because the use of a secondary or higher order of the diffracted light separates light into each order, and a high diffraction efficiency cannot be realized. In the present exemplary embodiment, the parameters of shape of the diffraction grating 2 are set such that the light quantity of the primary diffracted light among diffracted lights is maximized and the diffraction efficiency related to the primary diffracted light is maximized.

In a case where diffraction occurs in such a manner that the primary diffracted light outgoes into air from the diffractive optical element 1, a condition exists under which a secondary diffracted light 7 reflected on the surface on which the diffraction grating 2 formed occurs. In a case where the secondary diffracted light 7 occurs (exists), an equation of 2λ=p×(sin θ+n×sin θ3) is established, where the emitting angle (Littrow angle) of the primary diffracted light is θ, the emitting angle of a secondary reflection diffracted light in a case where the secondary reflection diffracted light 7 occurs is θ3, and the refractive index of the diffractive optical element 1 is n. On the other hand, θ is the emitting angle of the primary diffracted light 6, so that an equation of λ=2p×sin θ is established. The two equations produce sin θ3=3λ/(2n×p). If 0≦sin θ3≦1, a θ3 value exists, so that the secondary diffracted light 7 exists if a condition of 0≦3λ/(2n×p) 1 is satisfied. In other words, if 3λ/(2n×p)>1, the secondary diffracted light 7 does not exist. For this reason, it is required to set a pitch p to satisfy 2n×p<3λ so that a high diffraction efficiency is realized without causing the secondary diffracted light 7.

If a refractive index n of 1.45 and a wavelength λ of 855 nm are substituted for the above equation 2n×p<3λ, 2p (double of the pitch p) needs to be 844 nm or less, or 1131/mm or more in terms of the number of the ditches.

The above has described the diffraction of light emitting into air from the diffractive optical element 1. However, also in the diffraction of light falling on the diffractive optical element 1 from air, the second transmission diffracted light occurs under the same condition. Therefore, a condition of 2n×p<3λ needs to be similarly satisfied for a high diffraction efficiency.

In simulation, the number of the ditches of 1150 to 1500 was used in consideration of the above condition related to the pitch or the number of the ditches s. The calculation was performed while the grating depth D was changed from 1 μm to 3 μm and the duty ratio α was changed from 0.3 to 0.8. It is ineffective to increase the grating depth more than required or select a duty ratio largely deviating from 0.5, in terms of technique and cost in consideration of the production of the diffractive optical element, so that the grating depth D and the duty ratio a were set to the above range.

The diffraction efficiency acquired by calculation is a ratio of light quantity of the primary transmission diffracted light to total light quantity before transmission when transmission diffraction occurs in the diffraction grating 2 under the Littrow arrangement condition, and influence due to the surface opposite to the surface of the diffraction grating is not included. As described above, in general, there is no specific oscillation direction with regard to polarization in the light source for the SD-OCT and a high diffraction efficiency for both polarizations is required of the diffractive optical element. For this reason, an average diffraction efficiency for both polarizations was calculated.

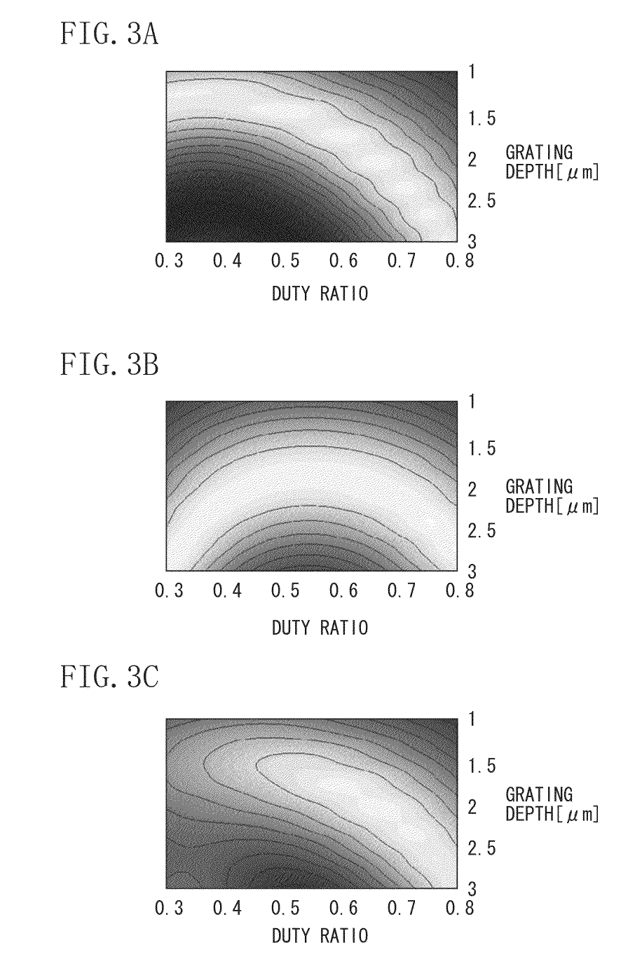

FIGS. 3A to 3C, FIGS. 4A to 4C, and FIGS. 5A to 5D illustrate calculation results. FIGS. 3A to 3C illustrate the diffraction efficiency for the grating depth D and the duty ratio a in a case where the number of grooves of the diffraction grating is 1200. FIG. 3A illustrates the diffraction efficiency for the TM polarization. FIG. 3B illustrates the diffraction efficiency for the TE polarization. FIG. 3C illustrates an average value of the diffraction efficiency for both polarizations. FIGS. 3A to 3C indicate that the value increases as black changes into white and a contour line is illustrated. As is clear from FIGS. 3A and 3B, the diffraction efficiency for the grating depth and the duty ratio differs according to polarization. In the case of the grating depth and the duty ratio acquired when both polarizations are high in diffraction efficiency, both polarizations are high in average diffraction efficiency.

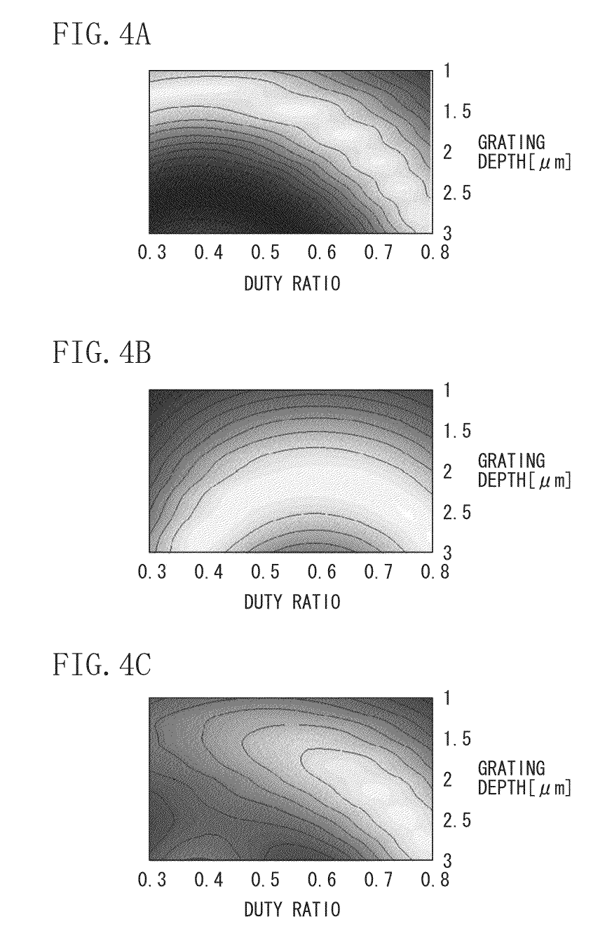

FIG. 4 is a chart illustrating the diffraction efficiency for the grating depth D and the duty ratio α in a case where the number of grooves of diffraction grating is 1400. FIG. 4A illustrates the diffraction efficiency for the TM polarization. FIG. 4B illustrates the diffraction efficiency for the TE polarization. FIG. 4C illustrates an average value of the diffraction efficiency for both polarizations. When FIG. 3A is compared with FIG. 4A, the dependence of the diffraction efficiency on the grating depth D and the duty ratio a is substantially kept unchanged for the TM polarization. However, when FIG. 3B is compared with FIG. 4B, the dependence of the diffraction efficiency on the grating depth D and the duty ratio a is different as for the TE polarization. Specifically, the grating depth when the diffraction efficiency is high in FIG. 4B tends to be greater than the grating depth when the diffraction efficiency is high in the diffraction efficiency in FIG. 3B. The same holds true of the duty ratio. The dependence of the average diffraction efficiency on the grating depth D and the duty ratio a also changes along with this. When FIG. 3C is compared with FIG. 4C, as is the case with the TM polarization, also with regard to the average diffraction efficiency, it is obvious that both of the grating depth D and the duty ratio a which are high in the diffraction efficiency increase according as the number of the ditches of the diffraction grating is increased.

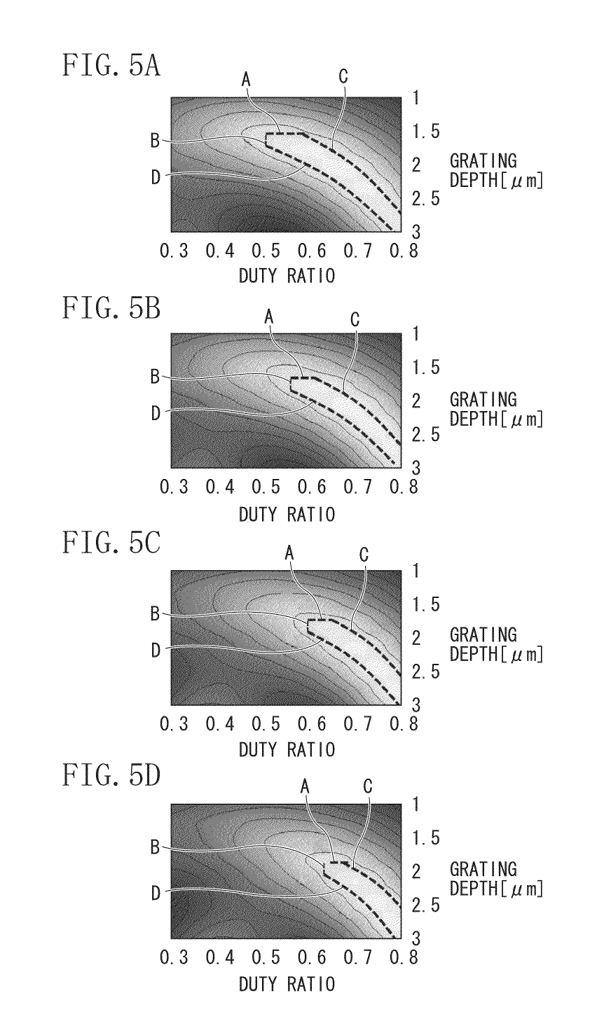

FIGS. 5A to 5D illustrate the dependence of the average diffraction efficiency on the grating depth and the duty ratio for both polarizations. FIG. 5A illustrates the dependence of the average diffraction efficiency on the grating depth and the duty ratio for both polarizations in a case where the number of the ditches of the diffraction grating is 1200. FIGS. 5B, 5C, and 5D illustrate the dependence thereof in a case where the number of the ditches of the diffraction grating is 1300, 1400, and 1500 respectively.

In general, the maximum diffraction efficiency of a conventional diffractive optical element is approximately 90% in a wavelength band to be used. For this reason, in the present exemplary embodiment, a diffraction efficiency of 93% is high enough as a calculation efficiency for realizing a diffraction efficiency higher than a conventional efficiency. In the present exemplary embodiment, therefore, we focus attention on the condition that the average diffraction efficiency for both polarizations is 93% or more and define the values of parameters satisfying the condition.

As results of surveying an area where the average diffraction efficiency for both polarizations is 93% or more, it can be seen from FIGS. 5A to 5D that the area can be defined by an area compassed by two straight lines A and B and two secondary curves C and D in calculation results for each of the numbers of the ditches of the diffraction grating. The straight line A defines the lower limit of the grating depth D satisfying the above condition and is defined by the grating depth D with a certain value. The straight line B defines the lower limit of the duty ratio satisfying the above condition and is defined by the duty ration a with a certain value.

The straight lines A and B have dependency on the number of the ditches of the diffraction grating. On the other hand, the secondary curves C and D have little dependency on the number of the ditches of the diffraction grating. Because the number of the ditches of the diffraction grating is a reciprocal of the pitch p, the straight line A is expressed as a function of the grating depth D and the pitch p and the straight line B is expressed as a function of the duty ratio a and the pitch p. The curves C and D are expressed as functions of the grating depth D and the duty ratio α.

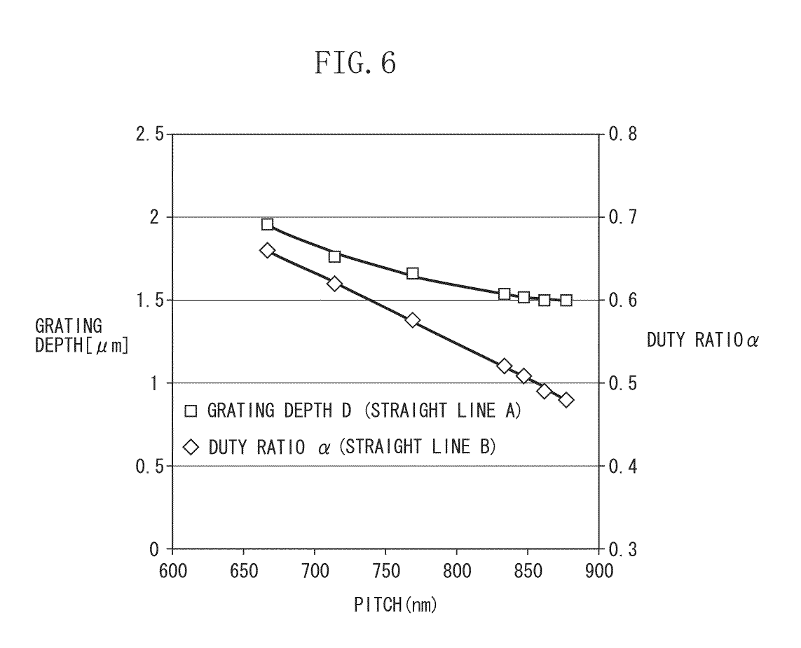

FIG. 6 is a chart in which the grating depth D (μm) defining the straight line A and the duty ratio a defining the straight line B are plotted relative to the number of the ditches of the diffraction grating, i.e., a reciprocal of the pitch p (nm).

If the grating depth D defining the straight line A is approximated by a quadratic function, a relationship between the grating depth D (μm) and the pitch p (nm) defining the straight line A can be expressed by the following equation 1:

D=7.8638×10⁻⁶×p²−1.4279×10⁻²×p+7.9734 (1).

If the duty ratio a defining the straight line B is approximated by a linear function, a relationship between the duty ratio a and the pitch p defining the straight line B can be expressed by the following equation 2:

α=−8.5747×10⁻⁴×p+1.2328 (2).

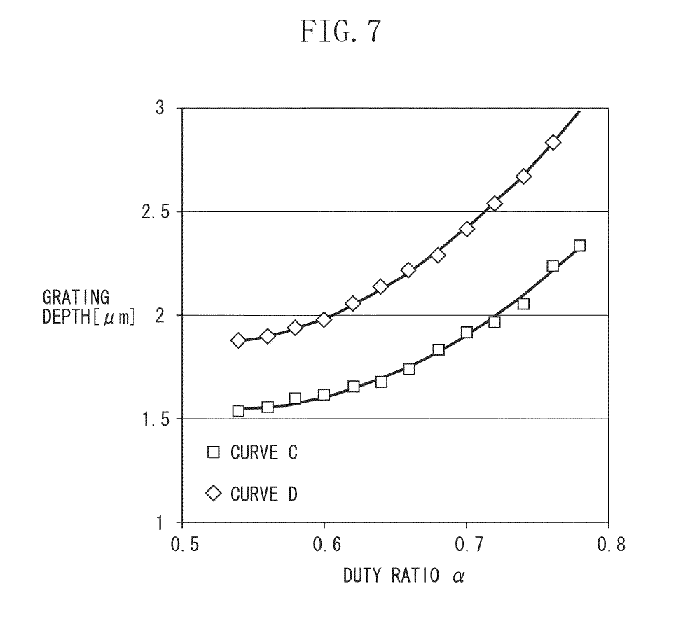

FIG. 7 is a chart illustrating a relationship between the grating depth D (μm) and the duty ratio α defining the curves C and D. If the relationship between the grating depth D (μm) and the duty ratio α is approximated by a quadratic function, a curve C can be expressed by the following equation 3:

C=13.19×α²−14.16×α+5.360 (3).

A curve D can be expressed by the following equation 4:

D=15.44×α²−15.73×α+5.870 (4).

As described above, the relation does not substantially depend on the number of the ditches of the diffraction grating.

The parameters of the diffraction grating capable of attaining the average diffraction efficiency of 93% or more for both polarizations need to satisfy the following five inequalities, where, inequalities 6 to 9 represent an area compassed by the straight lines A and B and the curves C and D:

2n×p<3A (5)

D>7.8638×10^{-6×}p²−1.4279×10^{-2×}p+7.9734 (6)

α>−8.5747×10⁻⁴×p+1.2328 (7)

D>13.19×α²−14.16×α+5.360 (8), and

D<15.44×α²−15.73×α+5.870 (9).

As described above, a high diffraction efficiency is required of the diffractive optical element for the SD-OCT throughout the wavelength band to be used. The above calculation was made using a wavelength of 855 nm. However, the following describes that a high diffraction efficiency can be achieved even in a wavelength band including wavelengths other than 855 nm as far as the shape of the diffraction grating satisfies the above inequalities.

As an example, the number of the ditches of the diffraction grating of 1200, a pitch of 833.3 nm, a grating depth of 2.06 μm, and a duty ratio of 0.66 are set as the parameters satisfying the above inequalities. The diffraction efficiency in this case was calculated. At the wavelength of 855 nm, the diffraction efficiency for the TE polarization is 97.6%, the diffraction efficiency for the TM polarization is 98.1%, and the average diffraction efficiency for both polarizations is 97.9%.

Although wavelength bands used in the SD-OCT are various in the vicinity of 800 nm, in the present exemplary embodiment, we assumed that light with wavelengths of 795 nm to 915 nm is falling, and the diffraction efficiency was calculated at the wavelength band. The incident angle of the light was 30.86 degrees that are the Littrow arrangement condition at the center wavelength of 855 nm.

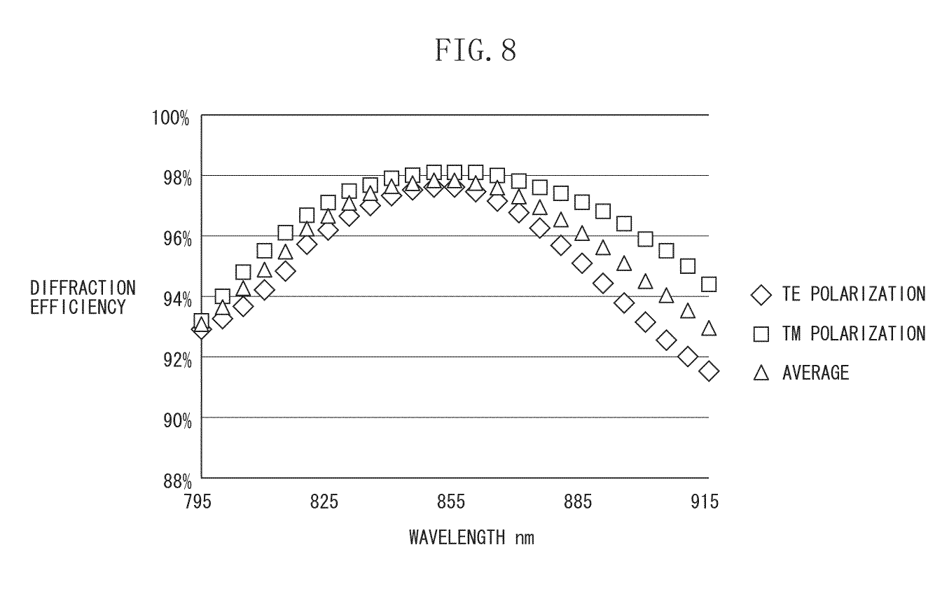

FIG. 8 illustrates results of the calculation of the diffraction efficiency at the wavelength band of 795 nm to 915 nm under the above condition. The average diffraction efficiency for both of the TE and TM polarizations at the edges of the wavelength band of 795 nm and 915 nm is 93.0%, which is high enough as compared with the diffraction efficiency at the same wavelength band, of a generally available conventional diffractive optical element. The parameters of the diffraction grating are thus set to change the diffraction efficiency as a quadratic function in which the efficiency value reaches a peak at a set wavelength 855 nm at the wavelength band. The parameters of the diffraction grating at which a high diffraction efficiency is obtained at the set wavelength of 855 nm are selected, and thereby, a high diffraction efficiency can be realized in the wavelength band to be used.

From the above, if the diffractive optical element has the diffraction grating defined by the three parameters of the pitch p or the number of the ditches of the diffraction grating, the grating depth D, and the duty ratio a satisfying the above inequalities, the diffractive optical element can realize a high diffraction efficiency for both of the TE and TM polarizations with a wavelength of 0.8 μm band.

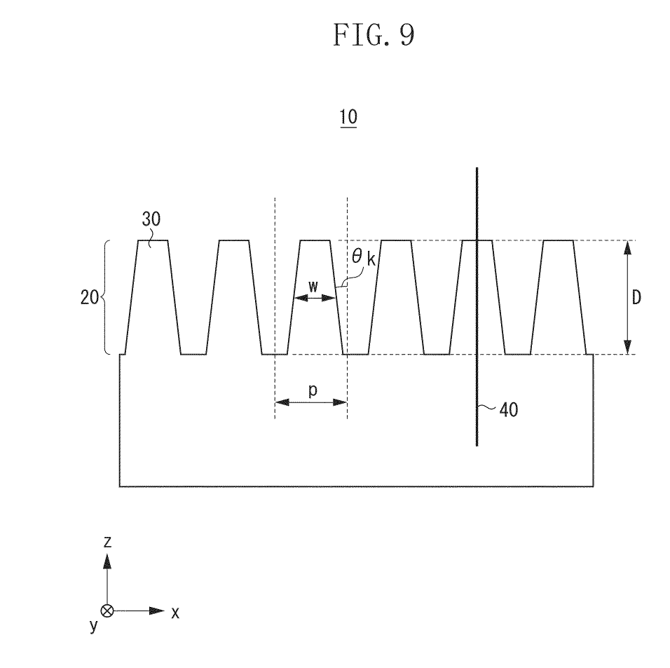

A diffractive optical element 10 according to a second exemplary embodiment will be described below. In the first exemplary embodiment, the cross section of the convex portion of the diffraction grating is rectangular. In the present exemplary embodiment, the cross section of the convex portion of the diffraction grating is not rectangular, and the side faces thereof are tilted. For example, if the diffraction grating is formed by etching, the side faces of the convex portion are formed not vertically but slantly. In this case, as illustrated in FIG. 9, a cross section of a convex portion 30 of a diffraction grating 20 is of an isosceles trapezoid shape. The side faces of the convex portion 30 are slanted with respect to a grating normal 40 and has an angle θk of several degrees. In other words, The width of the convex portion 30 changes in the depth direction of the diffraction grating 20. The pitch p and the grating depth D are similar to those in the first exemplary embodiment. A grating width w, however, is not constant in the direction of the grating normal 40, so that the grating width w is defined as a width in a direction in which the convex portion 30 is arranged (in the x direction) at the half position of the grating depth D. Thus, a duty ratio is determined using the width of the convex portion 30 in a predetermined depth of the diffraction grating 20.

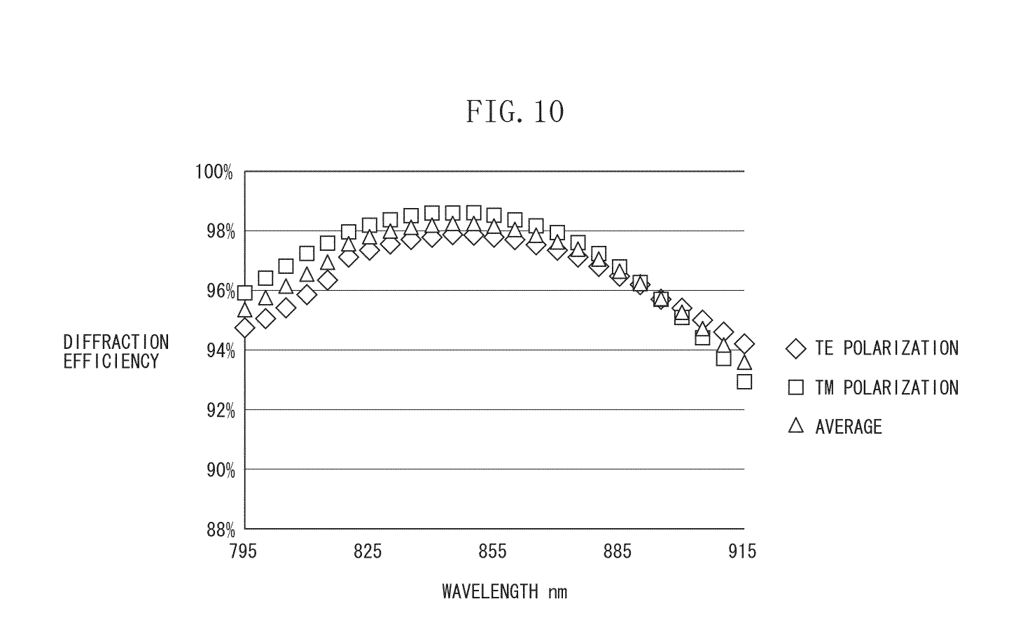

FIG. 10 illustrates results of calculating the dependence of the wavelength on the diffraction efficiency using the inclination angle θk of 4 degrees. As is the case with the calculation according to the first exemplary embodiment, the calculation was performed under the conditions of the number of the ditches of the diffraction grating of 1200, a pitch of 833.3 nm, a grating depth of 2.06 μm, a duty ratio of 0.66, and the incident angle of 30.86 degrees.

The average diffraction efficiency for both polarizations was 97.6% at a set wavelength of 855 nm, 94.7% at the edge of the wavelength band of 795 nm, and 93.0% at the edge of the wavelength band of 915 nm. Thereby, even if the side faces of the convex portion of the diffraction grating have a tilt angle, the diffractive optical element can realize a high diffraction efficiency for both of the TE and TM polarizations with a wavelength of 0.8 μm band as long as the diffractive optical element has the diffraction grating defined by the parameters satisfying the above inequalities.

A third exemplary embodiment describes a measuring device (an SD-OCT apparatus and an ophthalmologic apparatus) using the above transmission diffractive optical element and an optical coherence tomography method.

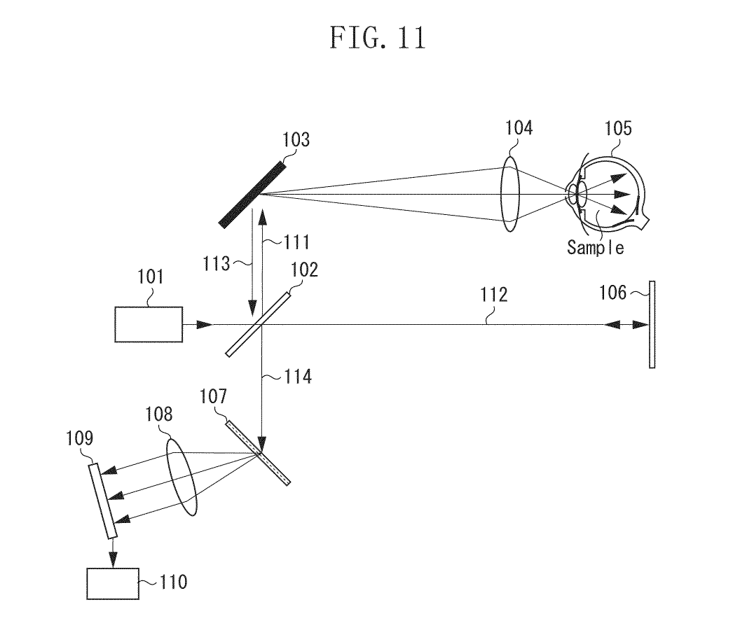

The configuration of the SD-OCT apparatus according to the present exemplary embodiment will be described below. FIG. 11 illustrates the configuration of the SD-OCT apparatus according to the present exemplary embodiment. Light having a wavelength of 0.8 μm band emitted from a light source 101 is split into a reference light 112 and a measurement light 111 by a beam splitter 102. The measurement light 111 with which an observation target (a subject: an eyeball 105) is irradiated through a lens 104 is returned as a return light 113 by reflection and scatter. The return light 113 and the reference light 112 are combined into an interfering light 114 by the beam splitter 102. The interfering light 114 is dispersed by a diffractive optical element 107 and passes through a lens 108, and a sensor 109 is irradiated therewith. The sensor 109 converts the intensity of the interfering light into an analog electric signal for each wavelength (photoelectric conversion), and outputs the analog electric signal to an image information processing unit 110. The image information processing unit 110 converts the input analog electric signal into a digital electric signal (A/D conversion), and acquires a tomographic image of a subject by calculation processing based on the digital electric signal for each wavelength. Specifically, the image information processing unit 110 applies processing such as Fourier transform processing to the digital electric signal for each wavelength to acquire a tomographic image of the eyeball 105. This enables examination of the fundus of the eyeball 105.

The transmission diffractive optical element according to the first or second exemplary embodiment is used for the diffractive optical element 107. The diffractive optical element 107 is used in the Littrow arrangement. The diffractive optical element according to the first or second exemplary embodiment can realize a high diffraction efficiency for both the TE and TM polarizations in a wavelength of 0.8 μm band, so that the resolution of an image acquired by the SD-OCT apparatus is decreased to allow high accuracy measurement.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-157609 filed Jul. 30, 2013, which is hereby incorporated by reference herein in its entirety.

What is claimed is:

1. A transmission diffractive optical element formed of quartz for a wavelength of 0.8 μm band,
wherein a refractive index of the quartz is n, a wavelength of light entering a diffraction grating is λ (nm), a pitch of the diffraction grating is p (nm), a depth of the diffraction grating is D (μm), and a duty ratio in which a width of the diffraction grating is divided by the pitch is ═,
wherein the pitch, the depth and the duty ratio of the diffraction grating satisfy following equations:
2n×p<3λ,
D>7.8638×10^{-6×}p²−1.4279×10^{-2×}p+7.9734α>−8.5747×10⁻⁴×p+1.2328,
D>13.19×α²−14.16×α+5.360, and
D<15.44×α²−15.73×α+5.870.

2. The transmission diffractive optical element according to claim 1, wherein
a light quantity of a primary diffracted light among lights diffracted by the diffraction grating is a maximum, and an emitting angle of the primary diffracted light in a certain wavelength is equal to an incident angle of light entering the diffraction grating.

3. The transmission diffractive optical element according to claim 1, wherein a center wavelength of light entering the diffraction grating is 840 nm to 880 nm.

4. The transmission diffractive optical element according to claim 1, wherein a cross section of the diffraction grating is of a trapezoid shape, and the duty ratio in which the width at a half position of the depth of the diffraction grating is divided by the pitch satisfies the equations.

5. The transmission diffractive optical element according to claim 1, wherein the width of the diffraction grating changes in a depth direction of the diffraction grating and the duty ratio in which the width of the diffraction grating at a predetermined depth of the diffraction grating is divided by the pitch satisfies the equations.

6. The transmission diffractive optical element according to claim 1, wherein the diffraction grating is rectangular in a cross section.

7. A measuring device for measuring a subject such that the subject is irradiated with light having a wavelength of 0.8 μm band, the measuring device comprising:
the transmission diffractive optical element according to claim 1 configured to disperse light from the subject; and
a sensor configured to detect the light dispersed by the transmission diffractive optical element.

8. The measuring device according to claim 7, wherein the transmission diffractive optical element is arranged in a Littrow arrangement.

9. The measuring device according to claim 7, wherein the measuring device is an optical coherence tomography.

10. The measuring device according to claim 9, wherein the optical coherence tomography is used for examining a fundus.
